(19) United States
(12) Patent Application Publication
(10) Pub. No.: US 2009/0163809 A1
(43) Pub. Date: Jun. 25, 2009

(54) Medical method and associated apparatus utilizable in accessing internal organs through skin surface

(76) Inventors: Scott D. Kane, Great Neck, NY (US); Peter J. Wilk, New York, NY (US); Timothy J. Nohara, Fonthill (CA)

Correspondence Address: R. Neil Sudol, 14 Colorado Avenue, Bridgeport, CT, 06605-1601, US

(21) Appl. No.: 10/859,763

(22) Filed: Jun. 3, 2004

(51) Int. Cl. A61B 8/14; G06F 15/00

(52) U.S. Cl. 600/443; 358/1.15

(57) ABSTRACT

A medical apparatus includes a scanner for generating raw image data of internal tissue structures of a patient, and a computer operatively connected to the scanner and programmed to derive a three-dimensional electronic map or model of the internal tissue structures from the raw data. An image reproduction device is operatively connected to the computer for reproducing the map or model in a visually readable format. The computer controls the image reproduction device to reproduce the map or model. The visually readable format includes graphical representations of the tissue structures alignable on a skin surface of the patient with the tissue structures. In use, a medical practitioner inserts a sharp instrument into a patient through the reproduced map, using the map as a locator assist.

### BACKGROUND OF THE INVENTION

This invention relates to a medical method and an associated apparatus utilizing a scanning device for obtaining three-dimensional structural data pertaining to internal tissue structures. The present method and apparatus utilize the three-dimensional data to provide a map of the internal tissue structures to assist the surgeon, anesthesiologist, radiologist, invasive cardiologist, or other medical personnel to access internal tissue structures at desired locations internal to a patient.

A long standing difficulty in routine medical practice is that of accessing an internal tissue structure such as a blood vessel or the spinal column through the skin surface. Typically, a physician or medical assistant uses a needle to puncture the skin of a patient, for purposes of injecting a therapeutic or diagnostic composition into a target tissue structure or to extract a sample of tissue for diagnostic purposes. In nearly every case, the medical personnel are operating blind, insofar as direct visual observation of the target internal tissue structures is not available. The person inserting a needle must rely chiefly on experience to form an educated judgment as to the location of the desired target point. Frequently, the individual medical practitioner must use trial and error to find the desired access point internal to the patient. Thus, the needle may be partially withdrawn and pivoted and then reinserted into the tissues. Needless to say, the conventional medical practice can be painful and traumatic to the patient.

It is generally impractical to use any of the several available scanning technologies to provide visual information as to internal tissue structures to guide a medical practitioner in accessing an internal organ or other tissue structure. The currently available scanning devices must generally be placed in contact with or near the patient in a manner that blocks effective access to the scanned site. In addition to being too cumbersome for many common patient access procedures, some of the currently available scanning technologies are simply too expensive to warrant widespread use for common needle directed or subdermal/hypodermic mediated operations. The least expensive scanning technology, ultrasound, that is currently on the market generates images that for most purposes have too low a resolution to be of effective assistance in placing a needle tip at a desired target location. There is however, an ultrasound technology not yet on the market that provides the requisite resolution in real time. The first production model, as currently contemplated, utilizes a scanning module that must be placed against the skin surface of the patient, which would block access to underlying tissue structures.

A need exists for a simple and inexpensive technique for enabling the utilization of three-dimensional structural data from a scanner in accessing sub-dermal tissue structures.

### OBJECTS OF THE INVENTION

A general object of the present invention is to provide a new technique and/or an associated apparatus for facilitating access to internal tissue structures.

A more specific object of the present invention is to provide such a technique and/or associated apparatus that provides three-dimensional structural data in a form useful to facilitate, for example, subdermal access to internal tissue structures.

It is another object of the present invention to provide such a technique and/or associated apparatus that is inexpensive and/or easy to use.

These and other objects of the present invention will be apparent from the drawings and descriptions herein. Although every object of the invention is attained in at least one embodiment of the invention, there is not necessarily any single embodiment that achieves all of the objects of the invention.

### SUMMARY OF THE INVENTION

A medical method in accordance with the present invention comprises scanning internal tissue structures of a patient to produce electronic data encoding the tissue structures, generating an electronically encoded map of the tissue structures, and reproducing the map in a visually readable format on a skin surface of the patient overlying the tissue structures so that graphical representations of the tissue structures in the readable format are aligned with corresponding ones of the tissue structures.

The map may be reproduced by printing the graphical representations. Preferably, the graphical representations are printed on a sheet or web which is then positioned on the skin surface of the patient. The printed sheet may be placed in direct contact with the skin of the patient overlying the desired access site. The printed sheet may be temporarily attached to the patient's skin, for instance, via a biocompatible adhesive. Thus, the printed sheet may be spaced from the skin surface by one or more intervening layers of different material.

Pursuant to another feature of the present invention, an accurate positioning of the sheet or web on the skin surface of the patient is accomplished in part by aligning the sheet or web with reference marks on the patient. These reference marks are typically made during the scanning procedure and depend on the relative position of the scanner with respect to the patient. The printed sheet or web may also be provided with locators or reference marks that are alignable with the reference marks provided on the patient during the scanning procedure.

In some cases, the printing of the graphical representations may be implemented directly on the skin surface of the patient, using a biocompatible ink. Preferably, the ink is water or alcohol soluble to facilitate a removal of the graphical representations after the access operation is completed.

Typically, the access operation includes puncturing the skin of the patient with a sharp instrument such as a hypodermic, epidural, or spinal needle. The distal end of the sharp instrument is preferably, although not necessarily, inserted through the printed map and more particularly through the sheet or web which carries the graphical representations of the patient's internal tissue structures. The medical practitioner generally inserts the instrument at the appropriate angle to the surface of the map at the puncture point.

Pursuant to a further feature of the present invention, the visually readable format of the tissue structure map includes an indicator of depth of at least one of the tissue structures. The indicator of depth may take one or more forms, including, but not limited to, a numerical symbol and a color. In the case of a numerical symbol, a number printed on the sheet or web or otherwise reproduced on the patient identifies the depth of an illustrated tissue structure, in a direction measured perpendicular to the skin surface of the patient. The number may appear next to or over the graphical representation of the illustrated tissue structure. Where color is used to indicate depth, a coding scheme is provided matching different colors with different distances below the patient's skin surface. Thus, a sub-dermal structure such as a vein that has a varying depth from the skin surface will have a succession of different colors along the length of the vein.

The visually readable format of the tissue structure map may be coded to identify tissue structures of different types. Color may be used to identify different types of tissue or different structures, instead of as an indicator of depth. For instance, in a map of the spinal column, one color may be used to represent bone, another color to represent cartilage, yet another color to represent connective tissue, and a further color to represent muscle tissue. These tissues may be automatically identified by the scanner computer in part by degrees of reflectivity and rates of waveform transmission (at least in the case of ultrasound).

The scanner computer may be programmed with pattern recognition software to determine the types of organs or tissue structures. In that case, the visually readable format of the tissue structure map may include an identification of at least one of the tissue structures. The identification may be accomplished by printing of a symbol, such as an alphanumeric designation or name. Alternatively, the identification may include a color.

It is to be noted that the present invention may find use with any type of scanning apparatus, provided that the resolution is sufficiently high to adequately map the tissue structure or structures to which access is desired. However, the scanning is preferably via ultrasound and includes transmitting ultrasonic pressure waves into the patient and detecting reflected ultrasonic waves from the internal tissue structures.

A medical apparatus in accordance with the present invention comprises a scanner for generating raw image data of internal tissue structures of a patient, and a computer operatively connected to the scanner and programmed to derive a three-dimensional electronic map or model of the internal tissue structures from the raw data. The medical apparatus further comprises an image reproduction device operatively connected to the computer for reproducing the map or model in a visually readable format. The computer is programmed to control the image reproduction device to reproduce the map or model. The visually readable format includes graphical representations of the tissue structures alignable on a skin surface of the patient with the tissue structures. The scanner, the computer, and the image reproduction device could be stand-alone, interconnected devices, partially integrated, or a fully integrated device. A fully integrated medical apparatus would have the benefit of being able to print the graphical representations directly on the skin surface during scanning, without requiring the movement of the scanner away from the skin in order to apply the graphical representations on the skin. In this preferred embodiment, reference marks are not needed.

The image reproduction device may include a printer that reproduces the map or model on a print substrate. The computer may be programmed to provide locator reference marks on the print substrate for assisting in positioning the print substrate on the skin surface of the patient.

The scanner may take the form of an ultrasound scanner. In this case, a fully integrated medical apparatus is achievable by employing scanner transducers directly on silicon (using, for example, micro electromechanical systems (MEMS) technology), which also includes the computer circuits needed to derive the three-dimensional electronic map or model of the internal tissue structures from the raw data. The integrated, image reproduction device would have to be acoustically transparent in order to support direct printing of the map onto the skin. Since the printed map need only have sufficient resolution to locate the underlying tissue structures for accessing them through the skin, various techniques, such as array thinning, known to those skilled in the art could be exploited to provide such transparency.

As discussed above, the visually readable format may includes an indicator of depth of at least one of the tissue structures, the computer being programmed to determine depth from the raw data and to generate the indicator of depth.

### BRIEF DESCRIPTION OF THE DRAWINGS

FIG. 1 is a block diagram of an apparatus in accordance with the present invention.

FIG. 2 is a schematic perspective view of a tissue structure map printed on a sheet or web by the apparatus of FIG. 1.

### DETAILED DESCRIPTION OF THE PREFERRED EMBODIMENTS

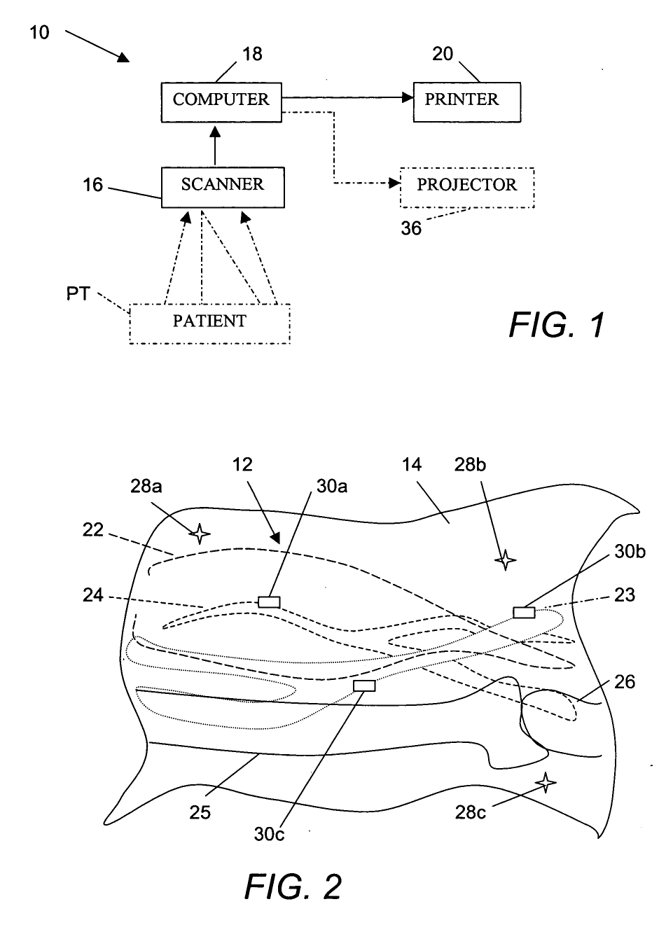

FIG. 1 schematically depicts an apparatus 10 for producing a visually readable map 12 (FIG. 2) of internal tissue structures of a patient PT exemplarily on a print substrate 14 such as a sheet, film, or web which is then placed over a patient at a desired location for assisting a medical practitioner in accessing a desired internal tissue structure of the patient. Apparatus 10 includes a scanner 16 such as an MRI apparatus, a CAT scanner, or an ultrasound scanner, for generating raw image data of internal tissue structures of a patient. A computer 18 is operatively connected to scanner 16 for deriving an electronic three-dimensional electronic map or model of the internal tissue structures from the raw data. Apparatus 10 further includes an image reproduction device 20 in the form of a printer that is operatively connected to computer 18 for reproducing the map or model in a visually readable format 12, as schematically depicted in FIG. 2. Computer 18 controls printer 20 to reproduce the electronic map or model in the visually readable format 12. Printed format 12 includes graphical representations 22-27 of scanned internal tissue structures. The print substrate 14 is disposable on a skin surface of the patient PT so that graphical representations 22-27 are aligned with the respective actual tissue structures.

Computer 18 is programmed to provide locator reference marks 28a, 28b, 28c on print substrate 14 in controlled locations with respect to graphical representations 22-27 so that the graphical representations 22-27 may be properly positioned in alignment with the respective internal tissue structures. To assist in positioning print substrate 14 on the skin surface of the patient PT, locator reference marks 28a, 28b, 28c are alignable with respective reference marks (not separately shown) formed on the skin surface of the patient during the scanning procedure. More particularly, the scanner may be provide with predetermined reference locations, such as corners of a carrier of a transducer array. Alternatively, the marks may be placed on the patient's skin surface by the operating personnel and an input device such as a mouse operated to advise computer 18 of the locations of the reference marks.

Scanner 16 preferably takes the form of an ultrasound scanner such as those disclosed in W07-367, 372, 274, 385, 420, and 428.

Visually readable format 12 of the electronic tissue structure map may include indicators 30a, 30b, 30c of tissue structure depth. Computer 18 is programmed to determine depth from the raw data from scanner 16 and to generate the indicators of depth. Indicators 30a, 30b, 30c may be numerical symbols or colors. The indicated depths correspond to distances below the skin surface along lines perpendicular to the skin surface. Where indicators 30a, 30b, 30c are numerical symbols, the numbers may appear next to or over the respective graphical representation 22-27. Where color is used to indicate depth, each graphical representation 22-27 may be illustrated in one or more colors, depending on the depth of the respective tissue structure. Computer 18 may have a display such as a computer monitor (not shown) which lists the depths coded by the different colors. Alternatively, a color brochure may be provided showing the coding scheme by which different colors are matched with different distances below the patient's skin surface. Thus, any graphical representation 22-27 may be have different colors or several different numerical indicators 30a, 30b, 30c along its respective length to communicate the varying depth of the corresponding tissue structure from the patient's skin surface.

In using the apparatus 10 of FIG. 1 to produce the visually readable format 12 of internal tissue structures of FIG. 2, scanner 16 generates outgoing waveforms 32 that are transmitted into the patient PT. Reflected waveforms 34 emanating from internal tissue structures of the patient PT are detected by scanner 16 to produce raw electronic data encoding the tissue structures. Scanner feeds the data to computer 18, which then generates an electronically encoded map of the tissue structures. Comptuer 18 controls printer 20 to reproduce the map in visually readable format 12 on print substrate 14 which is disposable on a skin surface of the patient overlying the scanned tissue structures so that graphical representations 22-27 of the tissue structures in the readable format 12 are aligned with the corresponding tissue structures.

Print substrate 14 is preferably placed in direct contact with the skin of the patient PT overlying a desired access site, such as a blood vessel, a spinal column, a skeletal joint, etc. The print substrate 14 may be temporarily attached to the patient's skin, for instance, via a biocompatible adhesive. The thickness of the print substrate 14, as well as the intervening adhesive layer, are known and tkaen into account by computer 18 in deriving depth indicators 30a, 30b, 30c.

Print substrate 14 is positioned on the skin surface of the patient PT in part by aligning reference marks 28a, 28b, 28c with corresponding reference marks (not shown) formed on the patient during the scanning procedure. The reference marks take into account the position of scanner 16 (and particularly waveform transmitters and sensors thereof) with respect to the patient.

After the placement of print substrate 14 on the patient PT, a medical practitioner punctures the skin of the patient with a sharp instrument such as a hypodermic needle (not shown) inserted through the print substrate 14 in a direction substantially perpendicular to the patient's skin surface. The practitioner inserts the instrument through a selected graphical representation 22-27 corresponding to a desired or target tissue structure, by an amount corresponding to the depth of the target tissue structure at the desired location. The depth is determined by attending to appropriate depth indicators 30a, 30b, 30c. Some interpolation may be necessary, particularly where the indicators 30a, 30b, 30c are numerical values that are all spaced from the desired access point. Alternatively, computer 28 may be provided with a monitor on which the visually readable map 12 is shown prior to the reproduction thereof on print substrate 14. The medical practitioner may use a mouse or other input device to inform computer 18 of a desired access point. Computer 18 then calculates the actual depth of the target tissue structure at the desired access point and shows the depth as part of the visually readable format 12, either on the computer monitor or display or on the printed version of the visually readable format 12 on print substrate 14.

Visually readable format 12 of the tissue structure map may be coded to identify tissue structures of different types, as represented in FIG. 2, by different types of lines for graphical representations 22-27. Color, line type, shading, cross-hatching, texture or other visual cues may be used to identify different types of tissue or different structures, instead of as an indicator of depth. For instance, in a map of the spinal column, one type of visual cue may be used to represent bone, another to represent cartilage, yet another to represent connective tissue, and a further to represent muscle tissue. These tissues may be automatically identified by the scanner computer in part by degrees of reflectivity and rates of waveform transmission (at least in the case of ultrasound).

Computer 18 may be programmed with pattern recognition software to determine the types of organs or tissue structures. In that case, the visually readable format 12 of the tissue structure map may include an identification of at least one of the tissue structures. The identification may be accomplished by printing of a symbol, such as an alphanumeric designation or name. Alternatively, the identification may include a color.

It is to be noted that the present invention may find use with any type of scanning apparatus, provided that the resolution is sufficiently high to adequately map the tissue structure or structures to which access is desired. However, the scanning is preferably via ultrasound and includes transmitting ultrasonic pressure waves into the patient and detecting reflected ultrasonic waves from the internal tissue structures.

The present invention may find use in any number of medical procedures, for example, interventional radiological procedures such as needle-directed tissue biopsies including but not limited to lung, kidney and liver procedures, both arterial and venous vasculature access operations, procedures for identifying and accessing the epidural and spinal space, and invasive cardiac procedures.

Although the invention has been described in terms of particular embodiments and applications, one of ordinary skill in the art, in light of this teaching, can generate additional embodiments and modifications without departing from the spirit of or exceeding the scope of the claimed invention. For example, in some cases, the printing of the graphical representations may be implemented directly on the skin surface of the patient, using a biocompatible ink. Preferably, the ink is water or alcohol soluble to facilitate a removal of the graphical representations after the access operation is completed.

As another example, the image reproduction device may take the form of a projector 36 that produces an image on the patient's skin or on a sheet placed over the skin.

Accordingly, it is to be understood that the drawings and descriptions herein are proffered by way of example to facilitate comprehension of the invention and should not be construed to limit the scope thereof.

What is claimed is:

1. A medical method comprising:
scanning internal tissue structures of a patient to produce electronic data encoding said tissue structures;
generating an electronically encoded map of said tissue structures; and
reproducing said map in a visually readable format on a skin surface of the patient overlying the tissue structures so that graphical representations of said tissue structures in said readable format are aligned with corresponding ones of said tissue structures.

2. The method defined in claim 1 wherein the reproducing of said map includes printing said graphical representations.

3. The method defined in claim 2 wherein the printing of said graphical representations is implemented on a sheet or web, the reproducing of said map further including positioning said sheet or web on the skin surface of the patient.

4. The method defined in claim 3 wherein the positioning of said sheet or web include aligning said sheet or web with reference marks on the patient.

5. The method defined in claim 4 wherein the scanning of said internal tissues includes placing a scanning device in juxtaposition to the patient, further comprising placing said reference marks on the patient in accordance with the position of said scanning device.

6. The method defined in claim 1 wherein said visually readable format includes an indicator of depth of at least one of said tissue structures.

7. The method defined in claim 6 wherein said indicator of depth includes a numerical symbol.

8. The method defined in claim 6 wherein said indicator of depth includes a color.

9. The method defined in claim 1 wherein said visually readable format includes an identification of at least one of said tissue structures.

10. The method defined in claim 9 wherein said identification includes a symbol.

11. The method defined in claim 9 wherein said identification includes a color.

12. The method defined in claim 1 wherein the scanning of said internal tissue structures includes transmitting ultrasonic pressure waves into the patient and detecting reflected ultrasonic waves from the internal tissue structures.

13. The method defined in claim 1, further comprising inserting a medical instrument into the patient after the reproducing of said map, said medical instrument being inserted into a selected one of said tissue structures shown as one of said graphical representations.

14. The method defined in claim 13 wherein said tissue structure is taken from the group consisting of a blood vessel and a spinal column.

15. The method defined in claim 1 wherein the reproducing of said map in a visually readable format on said skin surface includes printing said map directly on said skin surface.

16. A medical apparatus comprising:
a scanner for generating raw image data of internal tissue structures of a patient;
a computer operatively connected to said scanner and programmed to derive a three-dimensional electronic map or model of said internal tissue structures from said raw data; and
image reproduction means operatively connected to said computer for reproducing said map or model in a visually readable format, said computer being programmed to control said image reproduction means to reproduce said map or model, said visually readable format including graphical representations of said tissue structures alignable on a skin surface of the patient with said tissue structures.

17. The apparatus defined in claim 16 wherein said image reproduction means includes a printer.

18. The apparatus defined in claim 17 wherein said printer is integrated with said scanner so as to print said graphical representations directly on said skin surface.

19. The apparatus defined in claim 17 wherein said printer reproduces said map or model on a print substrate, said computer being programmed to provide locator reference marks on said print substrate for assisting in positioning said print substrate on the skin surface of the patient.

20. The apparatus defined in claim 16 wherein said scanner is an ultrasound scanner.

21. The apparatus defined in claim 16 wherein said visually readable format includes an indicator of depth of at least one of said tissue structures, said computer being programmed to determine depth from said raw data and to generate said indicator of depth.

22. The apparatus defined in claim 21 wherein said indicator of depth includes a numerical symbol.

23. The apparatus defined in claim 21 wherein said indicator of depth includes a color.

24. The apparatus defined in claim 16 wherein said visually readable format includes an identification of at least one of said tissue structures, said computer being programmed to identify said one of said tissue structures.

25. The apparatus defined in claim 23 wherein said identification includes a symbol.

26. The apparatus defined in claim 23 wherein said identification includes a color.
